(19) United States
(12) Patent Application Publication
(10) Pub. No.: US 2024/0166679 A1
(43) Pub. Date: May 23, 2024

(54) USE OF KETOACIDS FOR LIGNIN STABILIZATION DURING EXTRACTION FROM LIGNOCELLULOSIC BIOMASS

(71) Applicant: Lygg Corporation, Palo Alto, CA (US)

(72) Inventor: Graham Dick, Sunnyvale, CA (US)

(21) Appl. No.: 18/279,251

(22) PCT Filed: Mar. 4, 2022
(86) PCT No.: PCT/US22/19042

§ 371 Date: Aug. 29, 2023

Related U.S. Application Data
(60) Provisional application No. 63/157,594, filed on Mar. 5, 2021.

(51) Int. Cl. C07G 1/00; C12P 19/04

(52) U.S. Cl. CPC C07G 1/00; C12P 19/04

(57) ABSTRACT

A process which makes use of biodegradable ketoacids or ketoesters for producing a ketoacid or ketoester-stabilized, pure lignin with no residual sugars and no biomass fragments and enriched in carboxylic acids, carboxylic esters or ketones is provided. The disclosed stabilized lignin can be further modified after extraction or utilized as a source of renewable aromatic feedstock by hydrogenolysis.

### CROSS-REFERENCE TO RELATED APPLICATIONS

This application is a non-provisional application, and claims the benefit of provisional U.S. Patent Application No. 63/157,594, filed Mar. 5, 2021, which is hereby incorporated by reference it its entirety.

### FIELD

A process for stabilizing lignin during its extraction from lignocellulosic biomass is provided. The disclosed process makes use of biodegradable ketoacids and ketoesters, which are non-toxic and well-tolerated by the human body. Also disclosed is a stabilized lignin that can be further modified after extraction or utilized as a renewable source of reduced carbon.

### BACKGROUND

Lignin is a class of complex, heterogeneous organic polymers mainly found in the cell walls of plants and red algae together with cellulose and hemicellulose, and created from the in vivo polymerization of phenylpropanoids such as coniferyl, p-coumaryl and sinapyl alcohols. Due to the structure of these phenylpropanoids, the most frequent inter-monomeric linkage in lignin is the β-O-4 ether bond and lignin is enriched with syringyl, guaiacyl and 4-hydroxyphenyl monomers, which are aromatic compounds. Aromatic compounds are generally used in the production of a variety of chemicals and materials including plastics, drugs, cosmetics ingredients, and paints.

The rising level of atmospheric carbon dioxide calls for alternative strategies to mitigate and slow climate change. Lignocellulosic biomass is a massive source of renewable reduced carbon on earth. Over 80% of lignocellulosic biomass is composed of three major biopolymers—cellulose, hemicellulose, and lignin. These biopolymers can be separated and depolymerized into their constituent monomers, which include glucose from cellulose, predominantly xylose from hemicellulose, and aromatic molecules from lignin. However, while lignocellulosic biomass feedstocks are readily utilized for their cellulosic and hemicellulosic fractions either directly as materials or for their constituent monomers, the isolation and purification of lignin as a highly processable and upgradable material has been poorly developed.

Consequently, industrially produced lignin is mainly used as a source for fuel and less than 2% of all extracted lignin is utilized as a source for renewable chemicals or materials. This is because the presence of functional groups within lignin makes lignin a reactive polymer that degrades during extraction from biomass. Thus, the lignin that is extracted has lost its original structure because of the methodology that is used for its extraction from biomass.

Most existing industrial scale biomass valorization technologies focus on separating lignin from lignocellulosic biomass, such that purified cellulosic and hemicellulosic fractions may be obtained. In these processes, lignin is viewed as a contaminant due to its negative impact on the resulting product or subsequent processes.

The largest existing industrial scale biomass valorization processes are the pulp and paper processes, which produce purified cellulose fibers for papermaking. In the papermaking process, lignin is considered to negatively impact the quality of the final product, as lignin contributes to the yellowing of paper as it ages.

Similarly, emergent industrial scale biorefineries remove lignin because lignin can suppress the yields of glucose that can be obtained from the enzymatic hydrolysis of the cellulose. Consequently, these processes use harsh conditions to extract the lignin, which result in lignin degradation.

Under these harsh reaction conditions, the labile benzyl alcohols units in lignin's β-O-4 linkages are broken into reactive benzylic carbocations or alkenes. These species rapidly react with nearby electron-rich guaiacyl or syringyl lignin subunits in electrophilic aromatic substitution reactions. The resulting inter-unit C—C bonds prevent the lignin from being depolymerized efficiently by hydrogenolysis and inhibit its processibility.

Alternative economically viable solutions that generate renewable chemicals from lignin and allow optimized use of lignin in its entirety are therefore needed.

### SUMMARY

The present application presents a solution to the aforementioned challenges by providing quick, cost-effective and easily scalable processes for isolating and stabilizing lignin during biomass extraction. The disclosed processes make use of ketoacids or ketoesters to prevent lignin condensation and stabilize lignin during biomass fractionation, so that lignin can be modified after extraction. The resulting stabilized lignin may be further modified by exploiting the carboxylic acid, carboxylic ester, or ketone functionality contained in the ketoacid or ketoester or depolymerized into monomers that can be used as source of renewable feedstock for chemical and material manufacture or any other suitable application.

### BRIEF DESCRIPTION OF THE DRAWINGS

FIG. 1 is a diagram illustrating example formula(s) as used in some embodiments.

FIG. 2 is a diagram illustrating example formula(s) as used in some embodiments.

FIG. 3 is a diagram illustrating example formula(s) as used in some embodiments.

FIG. 4 is a diagram illustrating example formula(s) as used in some embodiments.

FIG. 5 is a diagram illustrating example formula(s) as used in some embodiments.

FIG. 6 is a diagram illustrating example formula(s) as used in some embodiments.

FIG. 7 is a diagram illustrating example compounds(s) as used in some embodiments.

FIG. 8 is a diagram illustrating example bifunctional ketones as used in some embodiments.

FIG. 9 is a diagram illustrating example formula(s) as used in some embodiments.

### DETAILED DESCRIPTION OF THE DRAWINGS

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In some embodiments, provided herein is a method for isolating and stabilizing lignin during biomass extraction. The disclosed method comprises: (i) obtaining lignocellulosic biomass; (ii) adding a ketoacid or ketoester, a solvent, and a catalytic quantity of a mineral or sulfonic acid to the lignocellulosic biomass to obtain a mixture; (iii) treating and filtering the mixture to produce a cellulose-free filtrate; and (iv) isolating lignin from the filtrate, thereby obtaining ketoacid or ketoester-stabilized lignin.

In some embodiments, the steps of treating and filtering the mixture to produce a cellulose-free filtrate comprises stirring and heating the mixture to a temperature from about 45° C. to about 165° C. for a time period between 5 minutes and 48 hours; cooling the mixture to room temperature; and filtering the mixture to produce a cellulose-free filtrate.

In some embodiments, the step of isolating lignin from the filtrate comprises exposing the filtrate to a temperature of about 45° C. or higher at a reduced pressure between about 2 mbar and about 100 mbar for a time period between 5 minutes and 24 hours with continuous stirring to remove the organic solvent and concentrate the filtrate; adding solvent to isolate the lignin; collecting the lignin by filtration and air-drying it; and subjecting the lignin to a temperature of about 45° C. or higher at a reduced pressure between about 2 mbar and about 100 mbar for a time period between 5 minutes and 24 hours to obtain ketoacid or ketoester-stabilized lignin.

The ketoacid or ketoester-stabilized lignin produced by the disclosed method is pure lignin, free of residual sugars and biomass fragments.

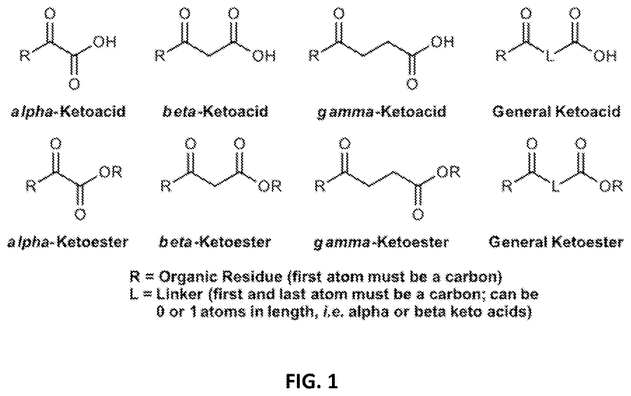

In some embodiments, the ketoacid or ketoester is an alpha-ketoacid, an alpha-ketoester, a beta-ketoacid, a beta-ketoester, a gamma-ketoacid, or a gamma-ketoester each respectively represented by a general formula as described in FIG. 1, wherein R is an organic residue and L is a linker.

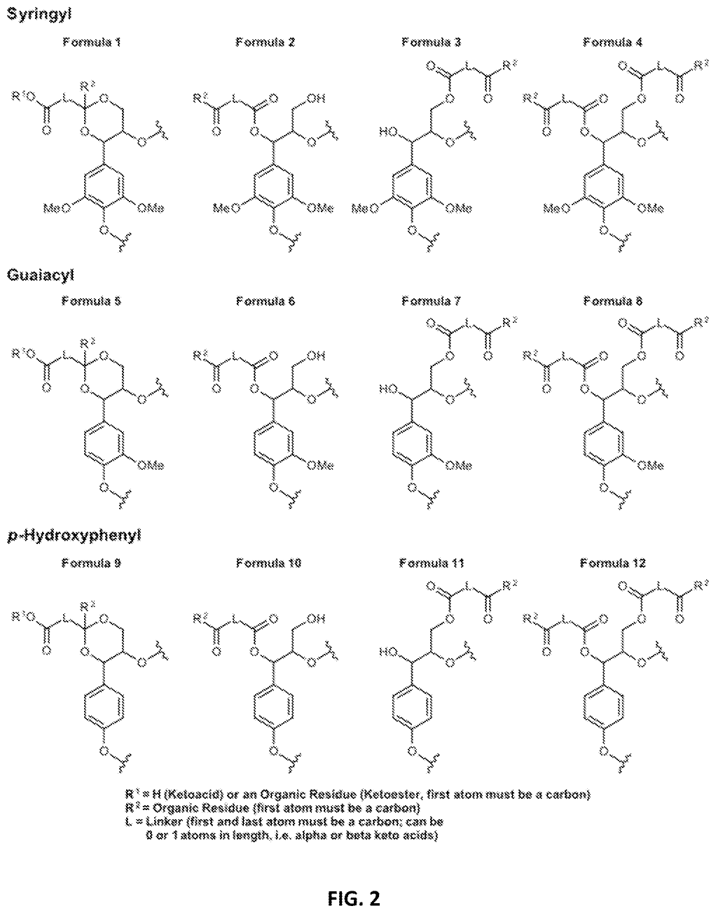

In some embodiments, the ketoacid or ketoester-stabilized lignin comprises stabilized syringyl, guaiacyl and/or p-hydroxyphenyl subunits, each respectively represented by one or more of formulae 1-12, wherein R¹ and R² are organic residues and L is a linker, as described in FIG. 2.

Suitable ketoacids and ketoesters include, but are not limited to, one or more of pyruvic acid, levulinic acid, acetoacetic acid, 2-oxobutyric acid, oxaloacetic acid, 2-oxovaleric acid, 3-oxopentanoic acid, 2-oxoglutaric acid, 3-oxoglutaric acid, 2-oxocaproic acid, 4-acetylbutyric acid, 6-oxoheptanoic acid, 2-oxooctanoic acid, 7-oxooctanoic acid, 5-oxoazelaic acid, 2-acetylbenzoic acid, 3-acetylbenzoic acid, 4-acetylbenzoic acid, methyl pyruvate, ethyl pyruvate, methyl levulinate, ethyl levulinate, propyl pyruvate, propyl levulinate, butyl pyruvate, and butyl levulinate.

In some embodiments, the solvent is an ether, a mixture of the ketoacid and water, or a mixture of the ketoester and water. In some embodiments, the ether is one or more of 1, 4-dioxane, tetrahydrofuran, 2-methyltetrahydrofuran, 3 -methyltetrahydrofuran, dimethoxyethane, cyclopentyl methyl ether, anisole, and bis(2-methoxyethyl) ether. In some embodiments the mixture of the ketoacid and water ranges in composition between 0% (v/v) water and 100% (v/v) water. In some embodiments the mixture of the ketoacid and water ranges in composition between about 20% (v/v) water and 30% (v/v) water. In some embodiments the mixture of the ketoester and water ranges in composition between 0% (v/v) water and 100% (v/v) water. In some embodiments the mixture of ketoester and water ranges in composition between about 0% (v/v) water and 10% (v/v) water.

Suitable mineral or sulfonic acids include, but are not limited to, one or more of hydrochloric acid, sulfuric acid, methane sulfonic acid, p-toluenesulfonic acid, nitric acid, hydrobromic acid, hydroiodic acid, perchloric acid, phosphoric acid, and hydrofluoric acid.

Additionally, provided herein is a ketoacid or ketoester-stabilized, pure lignin with no residual sugars and no biomass fragments.

In some embodiments, the disclosed ketoacid or ketoester-stabilized, pure lignin is produced by a method that comprises (i) obtaining lignocellulosic biomass; (ii) adding a ketoacid or ketoester, a solvent, and a catalytic quantity of a mineral or sulfonic acid to the lignocellulosic biomass to obtain a mixture; (iii) treating and filtering the mixture to produce a cellulose-free filtrate; and (iv) isolating lignin from the filtrate, thereby obtaining ketoacid or ketoester-stabilized lignin.

In some embodiments, the steps of treating and filtering the mixture to produce a cellulose-free filtrate comprises stirring and heating the mixture to a temperature from about 45° C. to about 165° C. for a time period between 5 minutes and 48 hours; cooling the mixture to room temperature; and filtering the mixture to produce a cellulose-free filtrate.

In some embodiments, the step of isolating lignin from the filtrate comprises exposing the filtrate to a temperature of about 45° C. or higher at a reduced pressure between about 2 mbar and about 100 mbar for a time period between 5 minutes and 24 hours with continuous stirring to remove the organic solvent and concentrate the filtrate; adding solvent to isolate the lignin; collecting the lignin by filtration and air-drying it; and subjecting the lignin to a temperature of about 45° C. or higher at a reduced pressure between about 2 mbar and about 100 mbar for a time period between 5 minutes and 24 hours to obtain ketoacid or ketoester-stabilized lignin.

The ketoacid or ketoester-stabilized lignin produced by the disclosed method is pure lignin, free of residual sugars and biomass fragments.

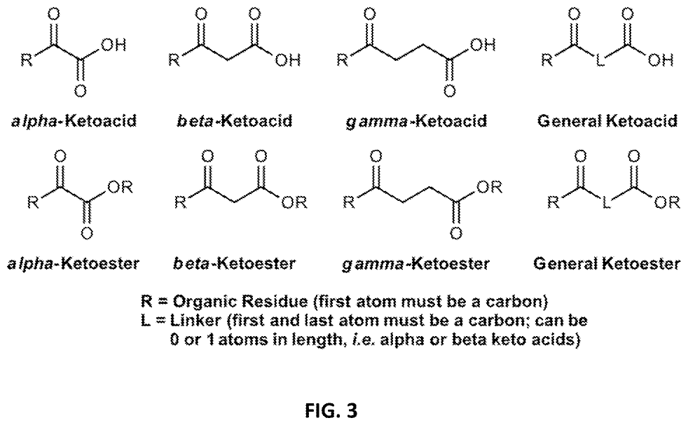

In some embodiments, the ketoacid or ketoester is an alpha-ketoacid, an alpha-ketoester, a beta-ketoacid, a beta-ketoester, a gamma-ketoacid, or a gamma-ketoester each respectively represented by a general formula as provided below, wherein R is an organic residue and L is a linker, as described by FIG. 3.

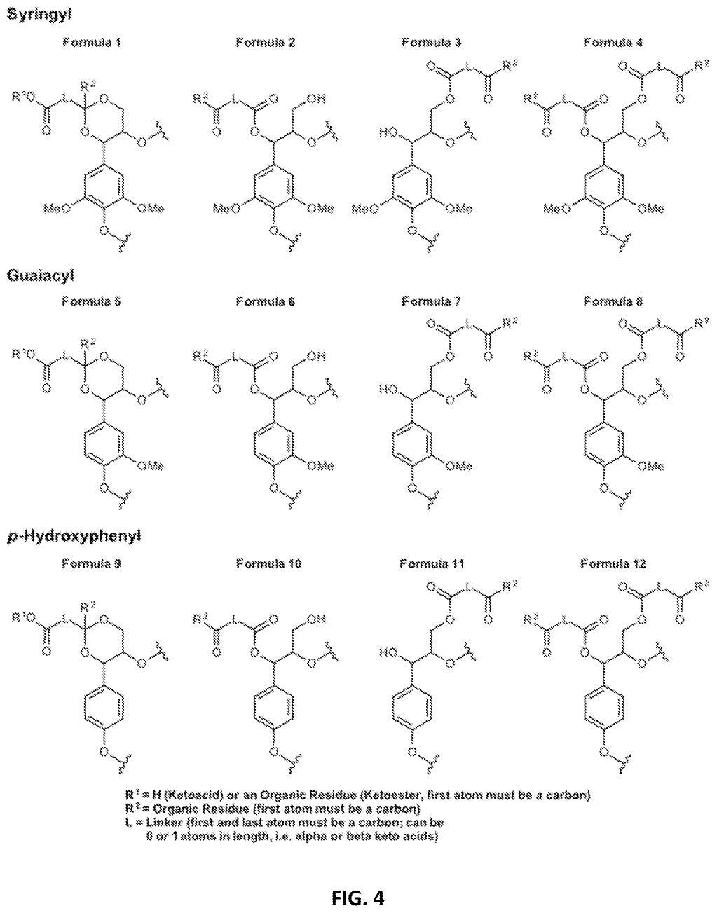

In some embodiments, the ketoacid or ketoester-stabilized lignin comprises stabilized syringyl, guaiacyl, and/or p-hydroxyphenyl subunits, each respectively represented by one or more of formulae 1-12, wherein R¹ and R² are organic residues and L is a linker, as described by FIG. 4.

Suitable ketoacids and ketoesters include, but are not limited to, one or more of pyruvic acid, levulinic acid, acetoacetic acid, 2-oxobutyric acid, oxaloacetic acid, 2-oxovaleric acid, 3-oxopentanoic acid, 2-oxoglutaric acid, 3-oxoglutaric acid, 2-oxocaproic acid, 4-acetylbutyric acid, 6-oxoheptanoic acid, 2-oxooctanoic acid, 7-oxooctanoic acid, 5-oxoazelaic acid, 2-acetylbenzoic acid, 3-acetylbenzoic acid, 4-acetylbenzoic acid, methyl pyruvate, ethyl pyruvate, methyl levulinate, ethyl levulinate, propyl pyruvate, propyl levulinate, butyl pyruvate, and butyl levulinate.

In some embodiments, the solvent is an ether, a mixture of the ketoacid and water, or a mixture of the ketoester and water. In some embodiments, the ether is one or more of 1, 4-dioxane, tetrahydrofuran, 2-methyltetrahydrofuran, 3 -methyltetrahydrofuran, dimethoxyethane, cyclopentyl methyl ether, anisole, and bis(2-methoxyethyl) ether. In some embodiments the mixture of the ketoacid and water ranges in composition between 0% (v/v) water and 100% (v/v) water. In some embodiments the mixture of the ketoacid and water ranges in composition between about 20% (v/v) water and 30% (v/v) water. In some embodiments the mixture of the ketoester and water ranges in composition between 0% (v/v) water and 100% (v/v) water. In some embodiments the mixture of the ketoester and water ranges in composition between about 0% (v/v) water and 10% (v/v) water.

Suitable mineral or sulfonic acids include, but are not limited to, one or more of hydrochloric acid, sulfuric acid, methanesulfonic acid, p-toluenesulfonic acid, nitric acid, hydrobromic acid, hydroiodic acid, perchloric acid, phosphoric acid, and hydrofluoric acid.

The foregoing and other features of the disclosure will become more apparent from the following detailed description of several embodiments, which proceeds with reference to the accompanying figure.

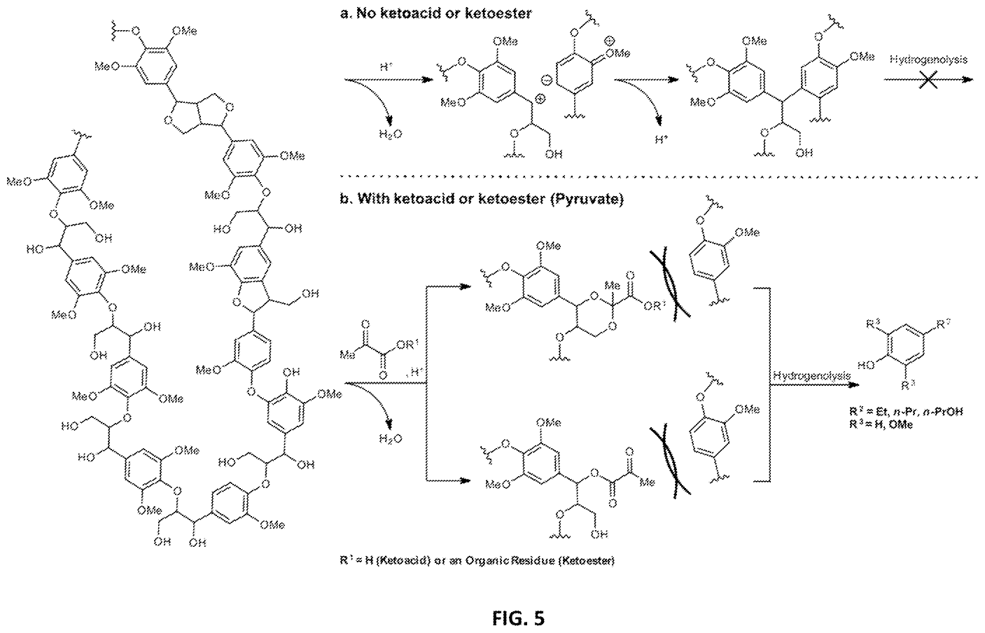

FIG. 5 compares routine extraction (a) of an exemplary lignin biopolymer containing electron-rich guaiacyl subunits, syringyl subunits, and the β-O-4 subunit (free diol), to extraction of the same lignin biopolymer in presence of ketoacids or ketoesters according to the disclosed process (b). In the absence of ketoacids or ketoesters (a), the benzyl alcohol of the β-O-4 subunit breaks down to produce a reactive carbocation that reacts with a nearby electron-rich guaiacyl subunit in an electrophilic aromatic substitution reaction, such that hydrogenolysis cannot cleave the carbon-carbon (C—C) bonds to produce lignin monomers (e.g. 4-propylsyringol, 4-propylguaiacol, 4-propylphenol). In presence of a ketoacid or ketoester (b), as the lignin is solubilized, the ketoacid or ketoester reacts with the lignin's β-O-4 subunit to form a stabilized ketal (1,3-dioxane structure) or ester. The ketal or ester prevents the formation of benzylic carbocations and the subsequent formation of inter-unit C—C bonds between lignin subunits, thus allowing the lignin to be depolymerized into lignin monomers by hydrogenolysis. The stabilized lignin subunits thus formed can be further modified following lignin's extraction allowing for the creation of novel materials, pharmaceuticals, and additives.

### DETAILED DESCRIPTION

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein.

Unless explained otherwise, all technical and scientific terms used herein have the same meaning as commonly understood to one of ordinary skill in the art to which this disclosure belongs. The materials, methods, and examples are illustrative only and not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−20% variation of the value it describes. Accordingly, in some embodiments, the numerical parameters set forth herein are approximations that can vary depending upon the desired properties for a particular embodiment. The recitation of ranges of values herein is merely intended to serve as a shorthand method of referring individually to each separate value falling within the range. To facilitate review of the various embodiments of this disclosure, the following explanations of specific terms are provided:
Analog: A compound having a structure similar to another, but differing from it, for example, in one or more atoms, functional groups, or substructure.
Carbocation: An ion with a positively charged carbon atom.
Contacting: Placing a substance in direct physical association with a material in solid, liquid, or gas form.
Control: A reference standard of a known value or range of values.
1, 3-Dioxane: A chemical compound having a saturated six-membered heterocycle with two oxygen atoms in place of carbon atoms in the 1- and 3-positions, and characterized by the molecular formula C₄H₈O₂.
Ester: A chemical functional group formed from the reaction of a carboxylic acid with an alcohol forming a structure with the following connectivity R¹C(O)(OR₂)), where the R groups are organic residues with the first atom being a carbon. The R groups can be equivalent or part of the same organic residue (e.g. ethyl acetate, γ-valerolactone).
Hybrid Material: A composite consisting of two or more components that are combined into a matrix at nanometer or molecular level. In some cases, one component is inorganic, and one component is organic.
Hydrogenolysis: A chemical reaction whereby a carbon-carbon or carbon-heteroatom single bond is cleaved by hydrogen. The heteroatom is usually oxygen, nitrogen or sulfur.
Ketal: A chemical functional group formed from the reaction of a ketone with alcohols forming a structure with the following connectivity R¹R²C(OR³)(OR⁴), where the R groups are organic residues with the first atom being a carbon. The R groups can be equivalent or part of the same organic residue (e.g. cyclohexanone, ethylene glycol).
Organic Residue: an atom or group of atoms that forms part of a molecule. The residue can be simple (e.g. a methyl group) or complex (e.g. a tetracyclic or a penicillin). There is no limitation on the size of the residue, its constituent atoms, or complexity. It can be represented by an “R” with or without a superscript, or by use of a bond drawn perpendicularly through a squiggly line.

### Process for Producing Stabilized Lignin from Lignocellulosic Biomass

Lignocellulosic biomass constitutes the bulk of terrestrial biomass and a relevant sustainable alternative to fossil carbon. Lignocellulosic biomass comprises three main biopolymers: cellulose, hemicellulose and lignin. Cellulose and hemicellulose are carbohydrate polymers containing five or six carbon sugar monomers, which are bound to lignin, an aromatic polymer that contains p-hydroxyphenyl, guaiacyl and syringyl subunits.

These biopolymers can be separated and depolymerized into their constituent monomers. Thus, cellulose depolymerization produces mainly glucose, hemicellulose depolymerization produces mainly xylose, and lignin polymerization produces aromatic monophenols. However, most lignin is used as a fuel and less than 2% of extracted lignin is used as a renewable resource, despite the great potential of lignin-derived materials and monophenols to be used as feedstocks for chemical and material manufacture, providing an alternative to petrochemicals. Such inefficient processing of lignin is mainly due to the harsh conditions by which lignin is extracted from the lignocellulosic biomass.

Lignin is a random polymer containing syringyl, guaiacyl and p-hydroxyphenyl units. The most abundant linkage in lignin is the β-aryl ether unit known as the β-O-4 linkage. Under harsh extraction conditions, the labile benzyl alcohols in the β-O-4 linkages produce reactive benzyl carbocations (referenced as FIG. 5 section a), which react with nearby electron-enriched guaiacyl and syringyl units in electrophilic aromatic substitution reactions. These reactions produce recalcitrant C—C bonds, which reduce the processability and upgradability of lignin and inhibit the further functionalization of the material after it has been extracted from the lignocellulosic biomass.

Provided herein is a process that overcomes the aforementioned challenges and drawbacks of traditional biomass extraction methods. The disclosed method calls for the addition of ketoacids or ketoesters to the lignin fractionation reactions, in order to stabilize the lignin during the extraction process. Lignin stabilization is achieved by allowing the ketones, carboxylic acids, or carboxylic esters in the ketoacid or ketoester molecules to react with the β-O-4 free diol units (referenced as FIG. 5, section b) and produce ketals or esters, which in turn prevents elimination of the benzyl alcohol and production of reactive benzylic carbocations, thereby stabilizing lignin.

The ketoacid or ketoester-stabilized lignin produced by the disclosed method has reactive carboxylic acid or carboxylic ester, and ketone functionality built in. Thus, the ketoacid's carboxylic acid or ketone functionality or the ketoester's carboxylic ester or ketone functionality can be further exploited to modify the lignin after extraction or interconverted into a large number of functionalities by chemical reactions. Since ketoacids and ketoesters exhibit low toxicity and are often human metabolites, the disclosed process produces a lignin material that is safe and presents no hazards. In addition, because of their low reactivity, ketoacids and ketoesters do not undergo acid-catalyzed aldol reactions to the same degree as aldehydes and, consequently, a low concentration of ketoacids or ketoesters may be used in the lignin extraction. Similarly, the ketoacids and ketoesters can be used as solvents for the lignin extraction.

Furthermore, addition of ketoacids to the biomass during extraction facilitates lignin purification, as the residual ketoacids and sugars are soluble in water, whereas lignin is water-insoluble. Thus, highly pure lignin can be easily isolated and produced in large-scale according to the disclosed method. The ketoacid-stabilized, pure lignin produced according to the disclosed method has no residual sugars and no biomass fragments, has great potential for post-extraction modification through the carboxylic acid and ketone functionality.

The stabilized lignin obtained by the disclosed method lacks inter-unit C—C bonds that form during the typical industrial biomass fractionation processes, and that ultimately produce very little lignin monomers. Rather, because of the absence of inter-unit C—C bond forming reactions, the stabilized lignin provided herein maintains its natural structure, has carboxylic acid, carboxylic ester, or ketone functionality, and, if desired, it is easily converted into lignin monomers by hydrogenolysis.

The disclosed method comprises: (i) obtaining lignocellulosic biomass; (ii) adding a ketoacid or ketoester, a solvent, and a catalytic quantity of a mineral or sulfonic acid to the lignocellulosic biomass to obtain a mixture; (iii) treating and filtering the mixture to produce a cellulose-free filtrate; and (iv) isolating lignin from the filtrate, thereby obtaining ketoacid or ketoester-stabilized lignin.

The step of treating and filtering the mixture to produce a cellulose-free filtrate comprises stirring and heating the mixture to a temperature from about 45° C. to about 165° C. for a time period between 5 minutes and 48 hours; cooling the mixture to room temperature; and filtering the mixture to produce a cellulose-free filtrate.

The step of isolating lignin from the filtrate comprises exposing the filtrate to a temperature of about 45° C. or higher at a reduced pressure between about 2 mbar and about 100 mbar for a time period between 5 minutes and 24 hours with continuous stirring to remove the organic solvent and concentrate the filtrate; adding solvent to isolate the lignin; collecting the lignin by filtration and air-drying it; and subjecting the lignin to a temperature of about 45° C. or higher at a reduced pressure between about 2 mbar and about 100 mbar for a time period between 5 minutes and 24 hours to obtain ketoacid-stabilized lignin.

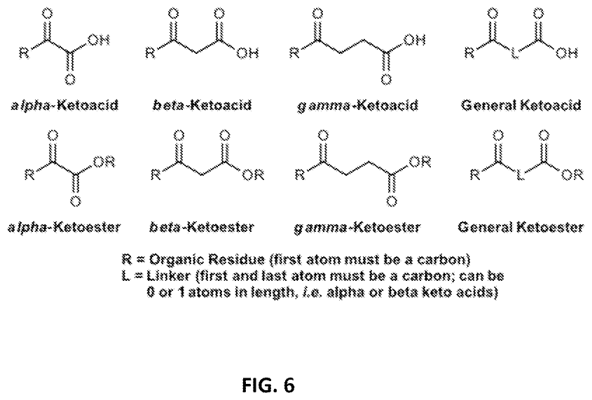

Suitable ketoacids or ketoesters that can be used in the disclosed method include, but are not limited to, alpha-ketoacids, alpha-ketoesters, beta-ketoacids, beta-ketoesters, gamma-ketoacids, and gamma-ketoesters each respectively represented by a general formula as provided below, wherein R is an organic residue and L is a linker, as described by FIG. 6.

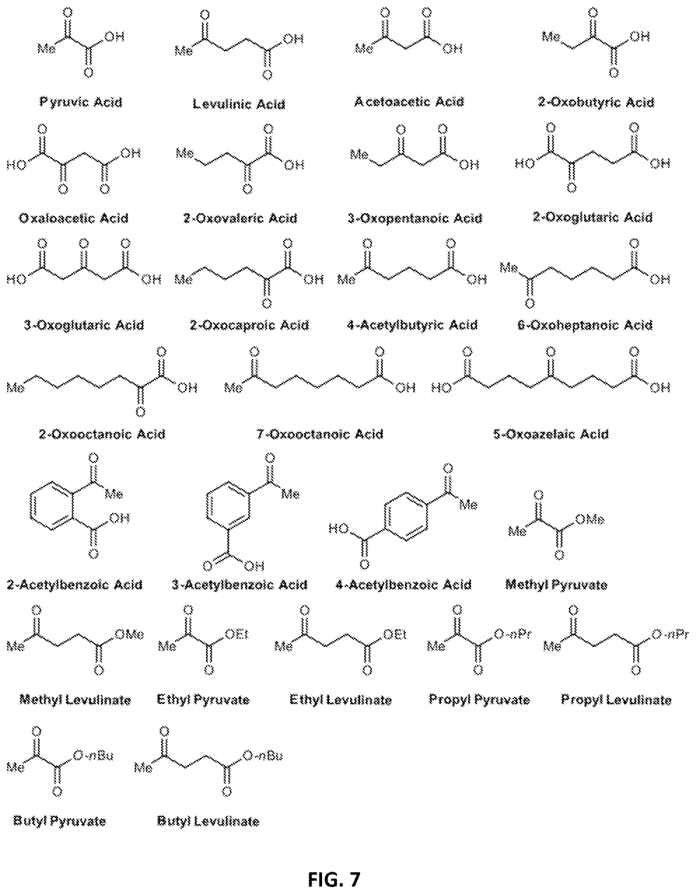

Exemplary suitable ketoacids and ketoesters that can be used in the disclosed method include, pyruvic acid, levulinic acid, acetoacetic acid, 2-oxobutyric acid, oxaloacetic acid, 2-oxovaleric acid, 3-oxopentanoic acid, 2-oxoglutaric acid, 3-oxoglutaric acid, 2-oxocaproic acid, 4-acetylbutyric acid, 6-oxoheptanoic acid, 2-oxooctanoic acid, 7-oxooctanoic acid, 5-oxoazelaic acid, 2-acetylbenzoic acid, 3-acetylbenzoic acid, 4-acetylbenzoic acid, methyl pyruvate, ethyl pyruvate, methyl levulinate, ethyl levulinate, propyl pyruvate, propyl levulinate, butyl pyruvate, and butyl levulinate. The structure of these compounds is represented by FIG. 7.

Suitable functional groups into which the ketoacids' or ketoesters' carboxylic acid, carboxylic ester or ketone functionalities can be converted or modified include, but are not limited to, alkene, alkyne, aldehyde, carboxylic acids, carboxylic ester, carboxylic amide, amino acids, ketene, ketone, diazoketone, imine, oxime, amine, acetal, ketal, hemi-acetal, hemi-ketal, fulminate, cyanate, isocyanate, isothiocyanate, nitrile, ether, thioether, hydroxyl, thiol, nitro, fluoride, chloride, bromide, iodide, azide, triflate, boronic acid, boronic acid ester, borate, borate salt, borane, silane, silyl ether, siloxane, silanol, sulfonamide, sulfonic acid, sulfonate, sulfoxide, sulfone, dithiane, phosphate, phosphate ester, phosphonate, phosphonic acid, phosphonate ester, phosphonium salt, phosphine, phosphite, phosphite ester, and phosphite salt, or a heterocycle selected from aziridine, 2H-azirine, oxirane, thiirane, azetidine, 2,3-dihydroazete, azete, 1,3-diazetidine, oxetane, 2H-oxete, thietane, 2H-thiete, azetidin-2-one, pyrrolidine,3-pyrroline, 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,3-triazole, tetrazole, tetrahydrofuran, furan, 1,3-diozolane, tetrahydrothiophene, thiophene, oxazole, isoxazole, isothiazole, thiazole, 1,2-oxathiolane, 1,3-oxathiolane, 1,2,5-oxadiazole, 1,2,3-oxadiazole, 1,3,4-thiadiazole, 1,2,5-thiadiazole, sulfolane, 2,4-thiazolidinedione, succinimide, 2-oxazolidone, hydantoin, piperidine, pyridine, piperazine, pyridazine, pyrimidine, pyrazine, 1,2,4-triazine, 1,3,5-triazine, tetrahydropyran, 2H-pyran, 4H-pyran, pyrylium, 1,4-dioxane, 1,4-dioxine, thiane, 2H-thiopyran, 4H-thiopyran, 1,3-dithiane, 1,4-dithiane, 1,3,5-trithiane, morpholine, 2H-1,2-oxazine, 4H-1,2-oxazine, 6H-1,2-oxazine, 2H-1,3 -oxazine, 4H-1,3-oxazine, 6H-1,3-oxazine, 4H-1,4-oxazine, 2H-1,4-oxazine, thiomorpholine, 4H-1,4-thiazine, 2H-1,2-thiazine, 6H-1,2-thiazine, 2H-1,4-thiazine, cytosine, thymine, uracil, thiomorpholine dioxide, hexahydro-1H-pyrrolizine, 1,4,5,6-tetrahydrocyclopental[b]pyrrole, 1,3a,4,6a-tetrahydropyrrolo[3,2-b]pyrrole, 1,4-dihydropyrrolo[3,2-b]pyrrole, 1,6-dihydropyrrolo[2,3-b]pyrrole, 6H-furo[2,3-b]pyrrole, 4H-furo[3,2-b]pyrrole, 4H-thieno[3,2-b]pyrrole, 6H-thieno[2,3 -b]pyrrole, 2,3 -dihydro-1H-indene, indene, indoline, 3H-indole, 1H-indole, 2H-isoindole, indolizine, 1H-indazole, benzimidazole, 4-azaindole, 5-azaindole, 6-azaindole, 7-azaindole, 7-azaindazole, pyrazolo[1,5-a]pyrimidine, purine, benzofuran, isobenzofuran, benzo[c]thiophene, benzo[b]thiophene, 1,2-benzisoxazole, 2,1-benzisoxazole, 1,2-benzisothiazole, 2,1-benzisothiazole, benzoxazole, benzthiazole, benzo[c][1,2,5]thiadiazole, 1,2-benzisothiazole-3(2H)-one, adenine, guanine, decahydroisoquinoline, decahydroquinoline, tetrahydroquinoline, 1,2-hydroquinoline, 1,2-dihydroisoquinoline, quinoline, isoquinoline, 4H-quinolizine, quinoxaline, phthalazine, quinazoline, cinnoline, 1,8-naphthyridine, pyrido[3,2-d]pyrimidine, pyrido[4,3-d]pyrimidine pyrido[3,4-d]pyrazine, pyrido[2,3-b]pyrazine, pteridine, 2H-chromene, 1H-isochromene, 3H-isochromene, 2H-chromen-2-one, 2H-benzo[e][1,2]oxazine, 2H-benzo[e][1,3]oxazine, 2H-benzo[b][1,4]oxazine, quinoline-2(1H)-one, isoquinolin-1(2H)-one, isoquinolin-1(2H)-one, fluorene, carbazole, dibenzofuran, acridine, phenazine, phenoxazine, phenothiazine, phenoxathiine, quinuclidine, 1-azaadamantane, 2-azaadamantane, 2,3-dihydroazepine, 2,5-dihydroazepine, 4,5-dihydroazepine, azepine, 2H-azepine, 3H-azepine, 4H-azepine, 1,2-diazepine, 1,3-diazepine, 1,4-diazepine, oxepane, thiepine, 1,4-thiazepine, azocane, azocine, thiocane, azonane and azecine.

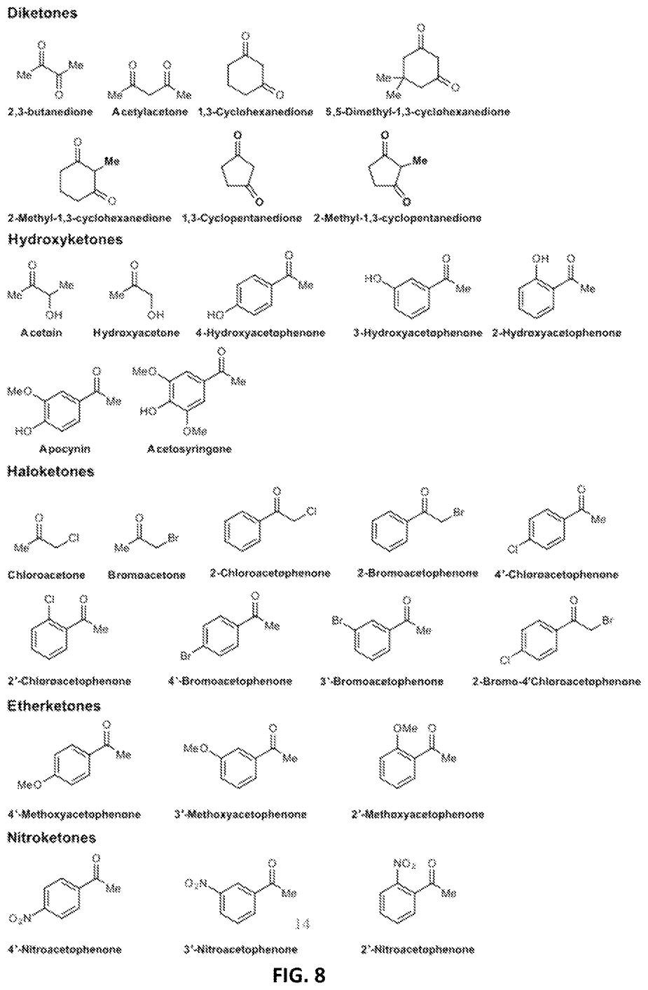

In some embodiments, the ketoacids or ketoesters in the disclosed method may be replaced by ketones, such as bifunctional ketones. Suitable bifunctional ketones include, but are not limited to, diketones, such as 2,3-butanedione, acetylacetone, 1,3-cyclohexanedione, 5,5-dimethyl-1,3-cyclohexanedione, 2-methyl-1,3-cyclohexanedione, 1,3-cyclopentanedione, 2-methyl-1,3-cyclopentanedione; hydroxy ketones, such as acetoin, 4-hydroxyacetophenone, 2-hydroxyacetophenone, 3-hydroxyacetophenon, hydroxyacetone, apocynin, and acetosyringone; haloketones, such as chloroacetone, bromoacetone, 2-chloroacetophenone, 2-bromoacetophenone, 4′-chloroacetophenone, 2′-chloroacetophenone, 4′-bromoacetophenone, 3′-bromoacetophenone, and 2-bromo-4′-chloroacetophenone; ether ketones, such as 4′-methoxyacetophenone, 3′-methoxyacetophenone, and 2′-methoxyacetophenone; and nitroketones, such as 4′-nitroacetophenone, 3′-nitroacetophenone and 2′-nitroacetophenone. The structures of some bifunctional ketones are provided in FIG. 8.

Additional suitable ketones include, but are not limited to, diketones, such as 1,2-cyclohexanedione, 1,4-cyclohexanedione, benzil, 1,2-cyclopentainedione, and 1,3-cyclopentanedione; haloketones, such as iodoacetone, 2-iodoacetophenone, 3′-chloroacetophenone, 2′-bromoacetophenone, 4′-iodoacetophenone, 3′-iodoacetophenone, and 2′-iodoacetophenone; ether ketones, such as methoxyacetone, and keto-amines, such as 4-aminoacetophenone, 3-aminoacetophenone, and 2-aminoacetophenone.

The amount of ketoacid or ketoester to be added to the fractionation mixture is in a range from about 1.0 to about 13.2 mmol/gram of biomass unless it used as a solvent.

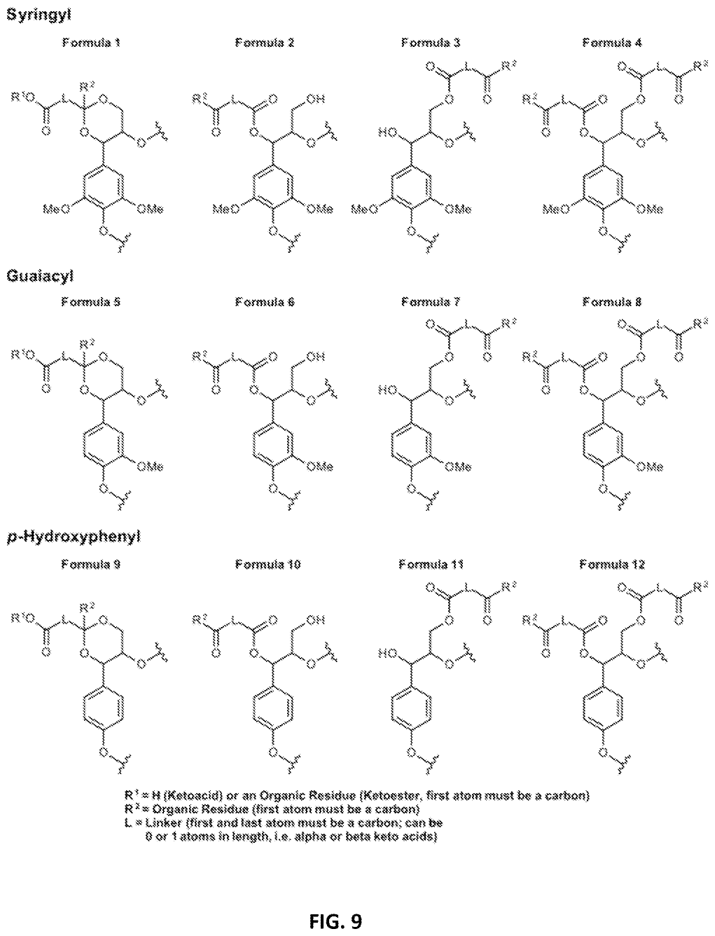

The ketoacid or ketoester-stabilized, pure lignin obtained by the disclosed method comprises stabilized syringyl, guaiacyl and/or p-hydroxyphenyl subunits, each respectively represented by one or more of formulae 1-12, wherein R¹ and R² are organic residues and L is a linker, as described by FIG. 9.

The solvent used in the fractionation mixture can be an ether, such as, for example, 1, 4-dioxane, or a mixture of the ketone, ketoacid, or ketoester and water, such as, for example, 70% (v/v) levulinic acid and 30% (v/v) water. The concentration of the solvent in the fractionation mixture is in a range from about 4 to about 10 mL/gram of biomass.

Lignin stabilization is optimized by the addition of a mineral or sulfonic acid to the fractionation mixture together with the ketoacid or ketoester, in a final concentration range from about 1 to about 10 mmol/gram of biomass. Suitable mineral or sulfonic acids include, but are not limited to, one or more of hydrochloric acid, sulfuric acid, methanesulfonic acid, p-toluenesulfonic acid, nitric acid, hydrobromic acid, hydroiodic acid, perchloric acid, phosphoric acid, and hydrofluoric acid.

The ketoacid or ketoester-stabilized lignin produced by the disclosed method is pure lignin, free of residual sugars and biomass fragments. The stabilized lignin obtained by the disclosed method can be further modified and/or formulated into compositions for the production of resins, adhesives, polymers, carbon fibers, insulation material, paints, surfactants, films, pigments, drug delivery substrates, powders, creams, sunscreen compositions, pharmaceuticals, explosives, flame-retardants, and the like.

The disclosed process presents several advantages over traditional separation processes, as it allows for further modification of the lignin after extraction, prevents aldol reactions that lead to inefficient lignin fragment stabilization, and it is environmentally friendly, since all products are fully biodegradable.

The following examples illustrate the disclosed method for producing stabilized lignin from lignocellulosic biomass, and how to obtain highly pure, ketoacid or ketoester-stabilized lignin, free of residual sugars and biomass fragments, according to the method presented herein.

### EXAMPLES

### Example 1: General Biomass Extraction Procedure

Hickory wood as debarked, dried wood chips (ca. 2 cm×4 cm×0.5 cm) were obtained. The wood chips were size reduced using a blender, such that the particle diameter was less than 6 mm. Size reduced hickory wood, which was a mixture of particle sizes, was used as is.

The wood biomass (2.0 g) was massed into a 40 ml vial equipped with a septum cap. To the vial was then added sequentially, a polytetrafluoroethylene (PTFE)-coated stir bar, a ketone (3.3-13.2 mmol per gram of biomass), 1,4-dioxane (4-10 ml of dioxane per gram of biomass), and hydrochloric acid (2-10 mmol per gram of biomass). The vial was sealed and heated to 85° C. and stirred at 700 RPM for three hours. The reaction was then cooled to room temperature (about 23° C.) and filtered through a funnel with a ground glass frit (medium porosity) to separate the cellulosic fraction. A quantitative transfer was performed using 1,4-dioxane (10 ml) and the cellulose was washed again with 1,4-dioxane (10 ml). The filtrate was transferred to a tared, 24/40, 250 ml, round bottom flask. The filtrate was then concentrated in vacuo using a rotoevaporator (45° C. bath temperature, 10 mbar ultimate pressure). Deionized water (50 ml) was added to precipitate the lignin. A PTFE-coated stir-bar was added, and the solution was stirred for 30 minutes at room temperature to break up any aggregates and ensure the full precipitation of the lignin from the concentrated filtrate. The stir-bar was then removed, and the precipitated lignin was collected by filtration through a nylon membrane filter (0.8 μm). The lignin was air-dried and returned to the tared, 24/40, 250 ml round bottom flask. The flask was then dried in vacuo on a rotoevaporator (45° C. bath temperature, 2 mbar ultimate pressure), to yield ketal-stabilized lignin as a powder.

### Example 2: Biomass Extraction with Pyruvic Acid

The procedure described in Example 1 was followed using hickory wood (2.0391 g), pyruvic acid (0.45 ml, 6.4 mmol, 1.5 equiv.), 1,4-dioxane (10 ml), and hydrochloric acid (0.35 ml, 4.2 mmol, 1.0 equiv.). The resulting lignin was isolated as light brown powder (0.4168 g, 20.4% weight).

### Example 3: Biomass Extraction with Levulinic Acid

The procedure described in Example 1 was followed using hickory wood (2.03753 g), levulinic acid (0.70 ml, 6.9 mmol, 1.6 equiv.), 1,4-dioxane (10 ml), and hydrochloric acid (0.35 ml, 4.2 mmol, 1.0 equiv.). The resulting lignin was isolated as light brown powder (0.4378 g, 21.1% weight).

### Example 4: Biomass Extraction with Oxaloacetic Acid

The procedure described in Example 1 was followed using hickory wood (2.0377 g), oxaloacetic acid (870.6 mg, 6.592 mmol, 1.6 equiv.), 1,4-dioxane (10 ml), and hydrochloric acid (0.35 ml, 4.2 mmol, 1.0 equiv.). The resulting lignin was isolated as light brown powder (0.4393 g, 22.0% weight).

### Example 5: Biomass Extraction with 2-Oxoglutaric Acid

The procedure described in Example 1 was followed using hickory wood (1.9965 g), 2-oxoglutaric acid (950.2 mg, 6.504 mmol, 1.6 equiv.), 1,4-dioxane (10 ml), and hydrochloric acid (0.35 ml, 4.2 mmol, 1.0 equiv.). The resulting lignin was isolated as light brown powder (0.4971 g, 24.9% weight).

These results indicate that addition of ketoacids during biomass fractionation significantly enhances the production of stabilized lignin and allows further modification of lignin for exploitation for renewable resources.

It should be recognized that illustrated embodiments are only examples of the disclosed product and methods and should not be considered a limitation on the scope of the invention. Rather, the scope of the invention is defined by the following claims. We therefore claim as our invention all that comes within the scope and spirit of these claims.

1. A method for isolating and stabilizing lignin during biomass extraction, wherein the method comprises:
(i) obtaining lignocellulosic biomass;
(ii) adding a ketoacid or ketoester, a solvent, and a catalytic quantity of a mineral or sulfonic
acid to the lignocellulosic biomass to obtain a mixture;
(iii) treating and filtering the mixture to produce a cellulose-free filtrate; and
(iv) isolating lignin from the filtrate, thereby obtaining ketoacid or ketoester-stabilized lignin.

2. The method of claim 1, wherein treating and filtering the mixture to produce a cellulose-free filtrate comprises stirring and heating the mixture to a temperature from about 45° C. to about 165° C. for a time period between 5 minutes and 48 hours; cooling the mixture to room temperature; and filtering the mixture to produce a cellulose-free filtrate.

3. The method of claim 2, wherein isolating lignin from the filtrate comprises exposing the filtrate to a temperature of about 45° C. or higher at a reduced pressure between about 2 mbar and about 100 mbar for a time period between 5 minutes and 24 hours with continuous stirring to remove the solvent and concentrate the filtrate; adding solvent to isolate the lignin; collecting the lignin by filtration and air-drying it; and subjecting the lignin to a temperature of about 45° C. or higher at a reduced pressure between about 2 mbar and about 100 mbar for a time period between 5 minutes and 24 hours to obtain ketoacid or ketoester-stabilized lignin.

4. The method of claim 3, wherein the ketoacid or ketoester-stabilized lignin is pure lignin, free of residual sugars and biomass fragments.

5. The method of claim 4, wherein the ketoacid or ketoester is an alpha-ketoacid, alpha-ketoester, beta-ketoacid, beta-ketoester, gamma-ketoacid, and gamma-ketoester each respectively represented by a general formula as provided below, wherein R is an organic residue and L is a linker:

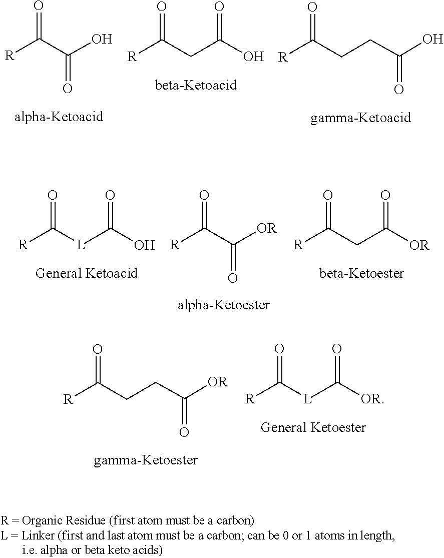

6. The method of claim 5, wherein the ketoacid or ketoester-stabilized lignin comprises stabilized syringyl, guaiacyl and/or p-hydroxyphenyl subunits, each respectively represented by Formula 1, Formula 2, Formula 3, Formula 4, Formula 5, Formula 6, Formula 7, Formula 8, Formula 9, Formula 10, Formula 11, and Formula 12 wherein R is an organic residue and L is a linker:

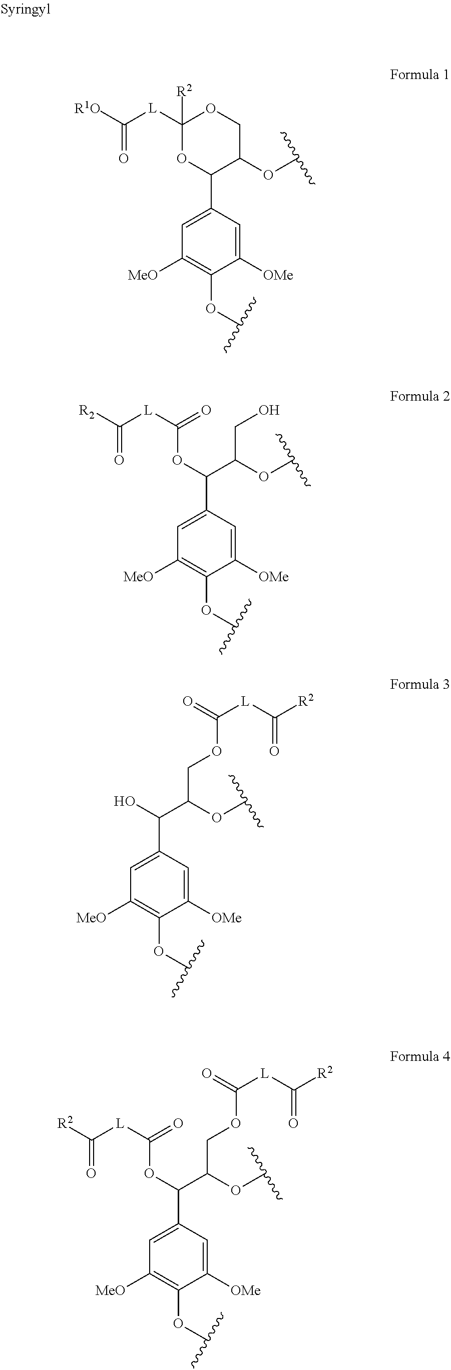

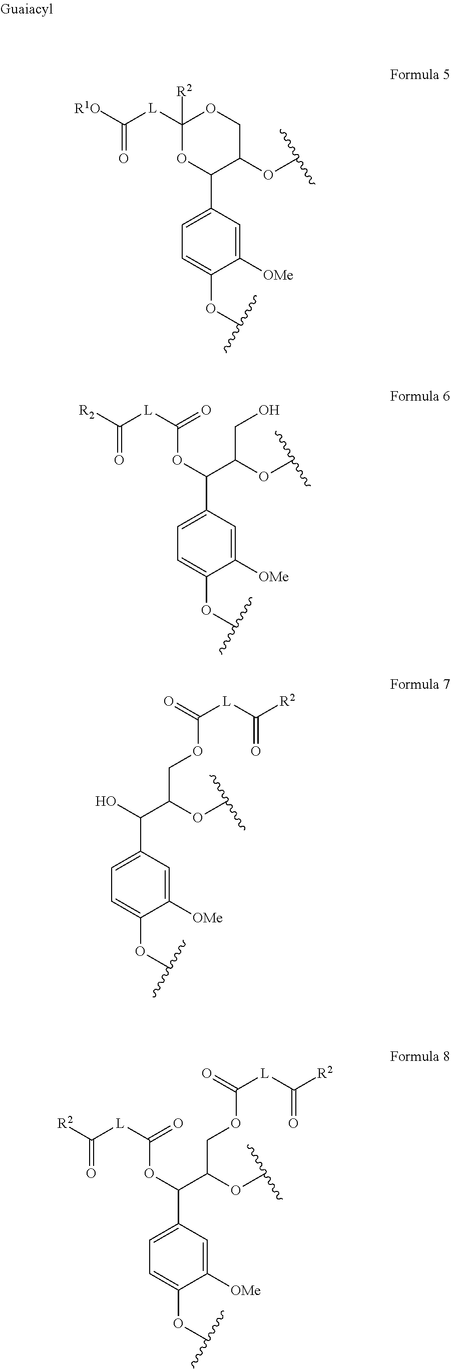

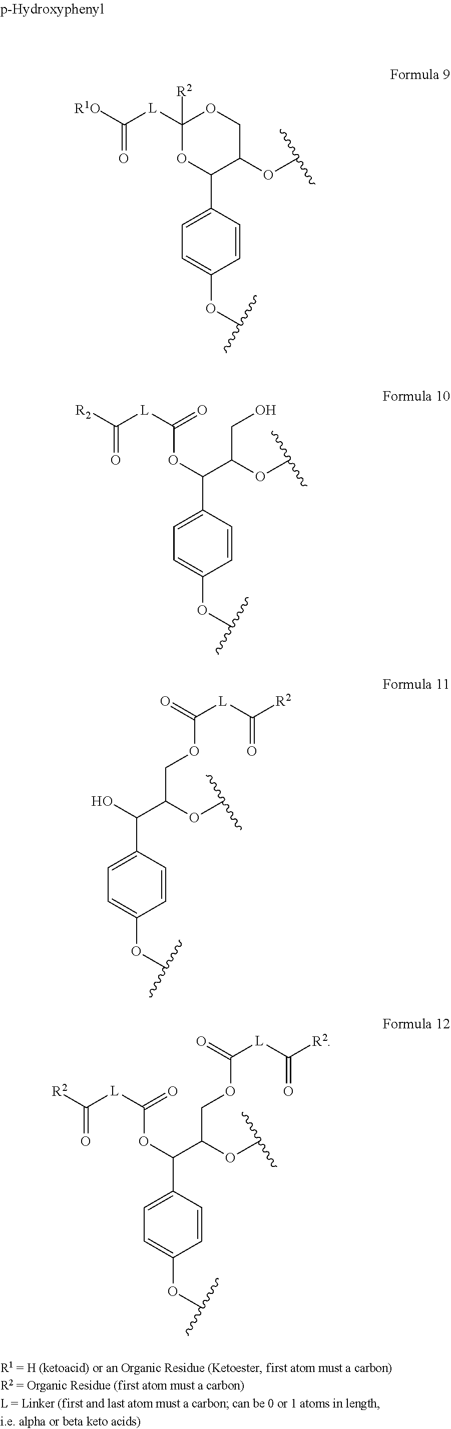

7. The method of claim 6, wherein the ketoacid or ketoester is one or more of pyruvic acid, levulinic acid, acetoacetic acid, 2-oxobutyric acid, oxaloacetic acid, 2-oxovaleric acid, 3-oxopentanoic acid, 2-oxoglutaric acid, 3-oxoglutaric acid, 2-oxocaproic acid, 4-acetylbutyric acid, 6-oxoheptanoic acid, 2-oxooctanoic acid, 7-oxooctanoic acid, 5-oxoazelaic acid, 2-acetylbenzoic acid, 3-acetylbenzoic acid, 4-acetylbenzoic acid, methyl pyruvate, ethyl pyruvate, methyl levulinate, ethyl levulinate, propyl pyruvate, propyl levulinate, butyl pyruvate, and butyl levulinate.

8. The method of claim 7, wherein the solvent is an ether, a mixture of the ketoacid and water, or a mixture of the ketoester and water.

9. The method of claim 8, wherein the ether is one or more of 1, 4-dioxane, tetrahydrofuran, 2-methyltetrahydrofuran, 3-methyltetrahydrofuran, dimethoxyethane, cyclopentyl methyl ether, anisole, and bis(2-methoxyethyl) ether; the mixture of the ketoacid and water ranges in composition between 0% (v/v) water and 100% (v/v) water; the mixture of the ketoacid and water ranges in composition between about 20% (v/v) water and 30% (v/v) water; the mixture of the ketoester and water ranges in composition between 0% (v/v) water and 100% (v/v) water; and the mixture of ketoester and water ranges in composition between about 0% (v/v) water and 10% (v/v) water.

10. The method of claim 9, wherein the mineral or sulfonic acid is one or more of hydrochloric acid, sulfuric acid, methanesulfonic acid, p-toluenesulfonic acid, nitric acid, hydrobromic acid, hydroiodic acid, perchloric acid, phosphoric acid, and hydrofluoric acid.

11. A ketoacid or ketoester-stabilized, pure lignin with no residual sugars and no biomass fragments.

12. The ketoacid or ketoester-stabilized, pure lignin of claim 11, wherein the ketoacid or ketoester-stabilized, pure lignin is produced by a method that comprises
(i) obtaining lignocellulosic biomass;
(ii) adding a ketoacid or ketoester, a solvent and a catalytic quantity of a mineral or sulfonic acid to the lignocellulosic biomass to obtain a mixture;
(iii) treating and filtering the mixture to produce a cellulose-free filtrate; and
(iv) isolating lignin from the filtrate, thereby obtaining ketoacid or ketoester-stabilized lignin.

13. The ketoacid or ketoester-stabilized, pure lignin of claim 12, wherein treating and filtering the mixture to produce a cellulose-free filtrate comprises stirring and heating the mixture to a temperature from about 45° C. to about 165° C. for a time period between 5 minutes and 48 hours; cooling the mixture to room temperature; and filtering the mixture to produce a cellulose-free filtrate.

14. The ketoacid or ketoester-stabilized, pure lignin of claim 13, wherein isolating lignin from the filtrate comprises exposing the filtrate to a temperature of about 45° C. or higher at a reduced pressure between about 2 mbar and about 100 mbar for a time period between 5 minutes and 24 hours with continuous stirring to remove the organic solvent and concentrate the filtrate; adding solvent to isolate the lignin; collecting the lignin by filtration and air-drying it; and subjecting the lignin to a temperature of about 45° C. or higher at a reduced pressure between about 2 mbar and about 100 mbar for a time period between 5 minutes and 24 hours to obtain ketoacid or ketoester-stabilized lignin.

15. The ketoacid or ketoester-stabilized, pure lignin of claim 14, wherein the ketoacid is an alpha-ketoacid, alpha-ketoester, beta-ketoacid, beta-ketoester, gamma-ketoacid, and gamma-ketoester, each respectively represented by a general formula, wherein R is an organic residue and L is a linker:

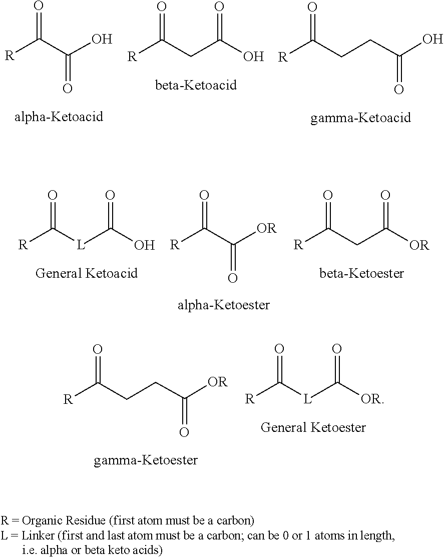

16. The ketoacid or ketoester-stabilized, pure lignin of claim 15, wherein the ketoacid or ketoester-stabilized lignin comprises stabilized syringyl, guaiacyl and/or p-hydroxyphenyl subunits, each respectively represented by one or more of formulae 1-12, wherein R¹ and R² are organic residues and L is a linker:

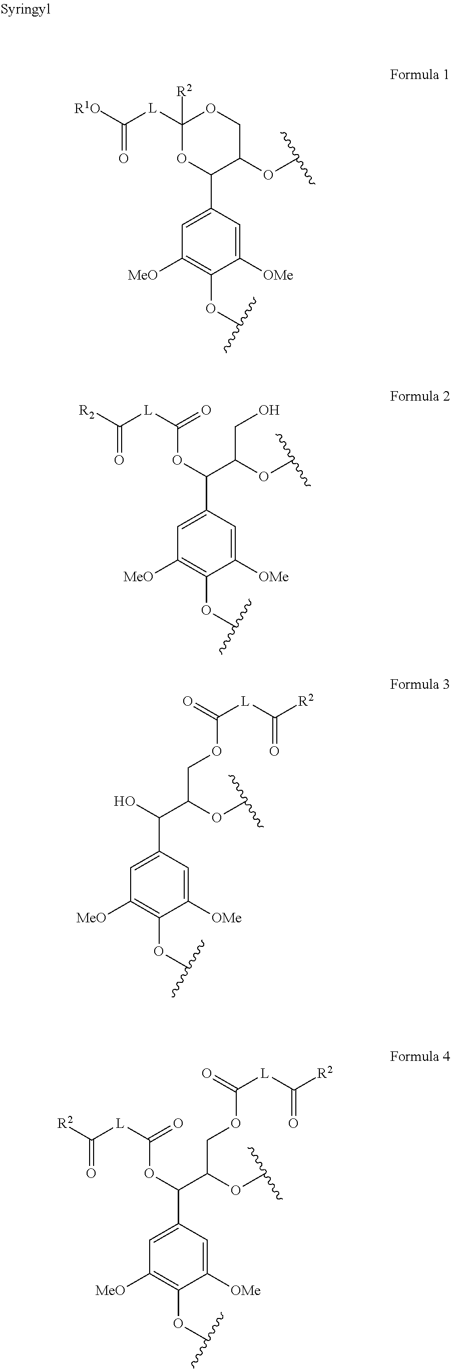

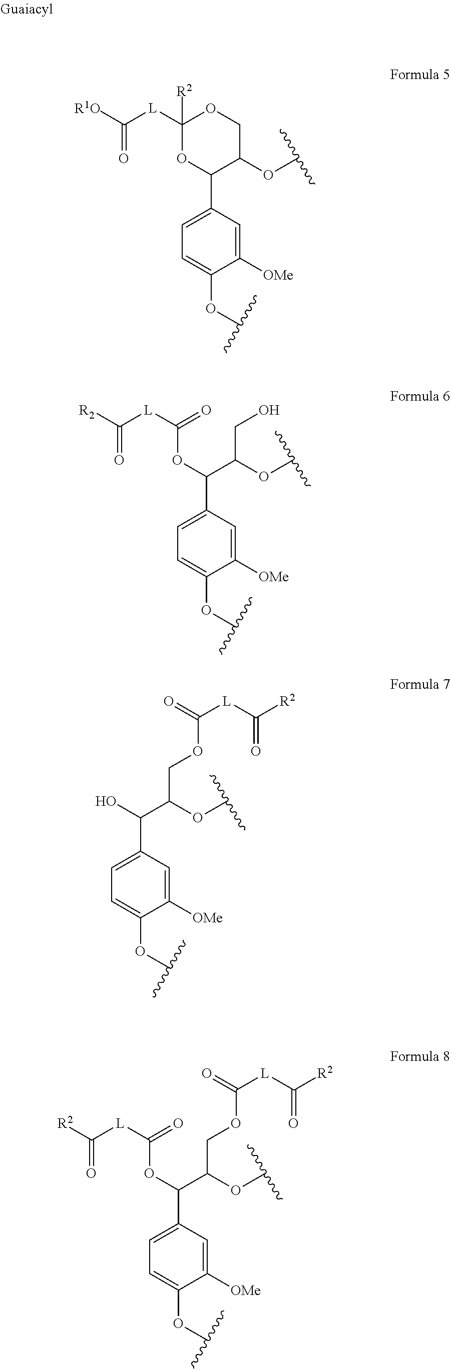

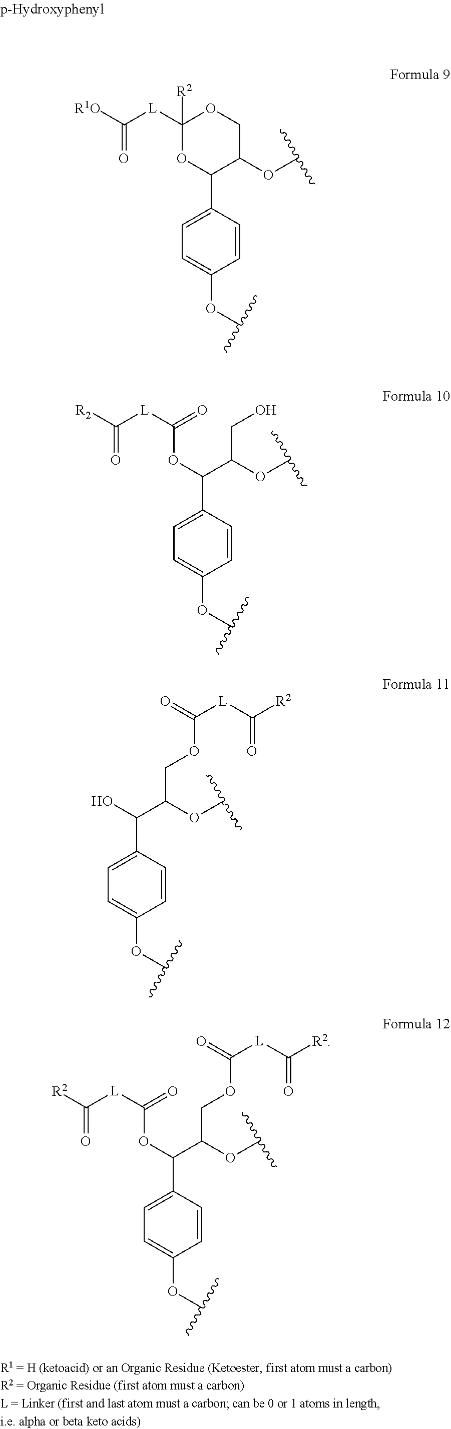

17. The ketoacid or ketoester-stabilized, pure lignin of claim 16, wherein the ketoacid or ketoester is one or more of pyruvic acid, levulinic acid, acetoacetic acid, 2-oxobutyric acid, oxaloacetic acid, 2-oxovaleric acid, 3 -oxopentanoic acid, 2-oxoglutaric acid, 3 -oxoglutaric acid, 2-oxocaproic acid, 4-acetylbutyric acid, 6-oxoheptanoic acid, 2-oxooctanoic acid, 7-oxooctanoic acid, 5-oxoazelaic acid, 2-acetylbenzoic acid, 3-acetylbenzoic acid, 4-acetylbenzoic acid, methyl pyruvate, ethyl pyruvate, methyl levulinate, ethyl levulinate, propyl pyruvate, propyl levulinate, butyl pyruvate, and butyl levulinate.

18. A composition comprising the ketoacid or ketoester-stabilized, pure lignin of claim 17.

19. The ketoacid or ketoester-stabilized, pure lignin of claim 18, wherein the solvent is an ether, a mixture of the ketoacid and water, or a mixture of the ketoester and water.

20. The ketoacid or ketoester-stabilized, pure lignin of claim 19, wherein the ether is one or more of 1, 4-dioxane, tetrahydrofuran, 2-methyltetrahydrofuran, 3-methyltetrahydrofuran, dimethoxyethane, cyclopentyl methyl ether, anisole, and bis(2-methoxyethyl) ether; the mixture of the ketoacid and water ranges in composition between 0% (v/v) water and 100% (v/v) water; the mixture of the ketoacid and water ranges in composition between about 20% (v/v) water and 30% (v/v) water; the mixture of the ketoester and water ranges in composition between 0% (v/v) water and 100% (v/v) water; and the mixture of ketoester and water ranges in composition between about 0% (v/v) water and 10% (v/v) water.

21. The ketoacid or ketoester-stabilized, pure lignin of claim 20, wherein the mineral or sulfonic acid is one or more of hydrochloric acid, sulfuric acid, methanesulfonic acid, p-toluenesulfonic acid, nitric acid, hydrobromic acid, hydroiodic acid, perchloric acid, phosphoric acid, and hydrofluoric acid.
